(19) United States
(12) Patent Application Publication
(10) Pub. No.: US 2024/0254067 A1
(43) Pub. Date: Aug. 1, 2024

(54) NOVEL METHOD FOR CONTINUOUS PHEROMONE PRODUCTION

(71) Applicant: MELCHIOR MATERIAL AND LIFE SCIENE FRANCE, LACQ (FR)

(72) Inventors: Olivier GUERRET, PERN (FR); Hugo LAUREANO, ARLES (FR)

(73) Assignee: MELCHIOR MATERIAL AND LIFE SCIENE FRANCE, LACQ (FR)

(21) Appl. No.: 18/564,580

(22) PCT Filed: May 27, 2022
(86) PCT No.: PCT/FR2022/051001

§ 371 Date: Nov. 27, 2023

(30) Foreign Application Priority Data
May 28, 2021 (FR) 2105616

(51) Int. Cl. C07C 45/38; B01J 31/18; B01J 31/22; C07C 45/80

(52) U.S. Cl. CPC C07C 45/38; B01J 31/1815; B01J 31/2208; C07C 45/80; B01J 2231/763; B01J 2531/004; B01J 2531/16

(57) ABSTRACT

The invention relates to a novel method for synthesising aldehyde-terminated pheromones according to the reaction: Where R is a linear aliphatic chain of formula CnH2n−2P+i where n is a natural number greater than or equal to 9 and p is an integer between 1 and 4. This method is characterised in that it is carried out continuously in a polar solvent in the presence of a copper-based catalytic system under an air pressure of more than 1 bar and at a temperature of between 30 and 200° C.

Novel method for synthesizing aldehyde-terminated pheromones according to the reaction:

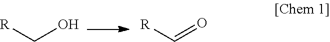

where R is a linear aliphatic chain of formula CₙH₂ₙ₋₂ₚ₊₁ where n is a natural number greater than or equal to 9 and where p is an integer between 1 and 4. This method is characterized in that it is carried out continuously in a polar solvent in the presence of a copper-based catalytic system under an air pressure of more than 1 bar and at a temperature of between 30 and 200° C. This method offers the advantage of high productivity and high selectivity of the reaction.

Insect pheromones are the specific communication tools of each species. For this communication to be effective, the mixture of chemical compounds, called a pheromonal bouquet, must be very precise. Thus, the sex pheromone of cydalima perspectalis, commonly known as the box tree moth, is a mixture of Z and E-11 hexadecenal in the precise ratio of 80/20. Outside of this ratio the male is not properly attracted to an attractant. Moreover, traces of Z/E-11-hexadecenol completely neutralize the effect of the pheromone.

Moreover, the synthesis of pheromones for industrial applications is part of a regulatory framework which drastically limits the possibility of producing pheromones with impurities in uncontrolled number and amount.

These two constraints induce significant technological challenges for the person skilled in the art, especially since these molecules are used in occasional communications between individuals of the same species, which induces rapid degradation of the molecules once emitted into the atmosphere. This fragile nature necessarily increases the appearance of impurities in a synthesis method. This is particularly the case for sex pheromones of Lepidoptera whose main components have compounds of general formulas:

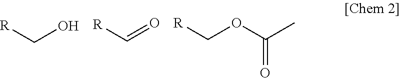

where R is a linear aliphatic radical including between 1 and 4 unsaturations in the form of a conjugated double or triple bond or not. The general formula of these linear groups R can therefore be written: CₙH₂ₙ₋₂ₚ₊₁ where n is an integer greater than 9 and p, which represents the number of unsaturations, is an integer between 1 and 4 (a triple bond counting for 2 unsaturations).

As the person skilled in the art easily anticipates, the terminal alcohols R—CH₂OH are the precursors of aldehydes or acetates. While the acetylation of alcohols can easily be done by reacting the alcohol with an acetylation agent such as acetic anhydride or acetyl chloride in the presence of a base such as a tertiary amine, the manufacture of aldehydes is more complicated.

Indeed, the known methods for obtaining the aldehydes of this family are first of all oxidations in the presence of organic peracids (chloro-perbenzoic, peracetic, perpropionic acid). These oxidants are aggressive and also attack the unsaturations present on the fatty chain, generating numerous impurities such as epoxides or oligomers. Moreover, fatty acid overoxidation is difficult to avoid. Another method consists in using a sodium hypochlorite or a sodium hypobromite in the presence of a nitroxide or a nitroxonium salt. This method allows to avoid overoxidation but chlorinations or brominations of unsaturations are observed which are very troublesome from a regulatory point of view. To limit this effect, bleach can be replaced by iodobenzene peracetate but sacrificing the economic efficiency of the synthesis.

Moreover, all these methods are methods carried out in batches, which requires very large reaction volumes.

It is therefore important to successfully industrialize pheromones of the RCH2O form to find a productive and selective oxidation technology.

Only two patents report attempts to couple the synthesis of pheromones and the concepts of continuous chemistry. The first work is reported in patent U.S. Pat. No. 9,789,455B2. In this patent, the inventors have described continuous synthesis equipment for producing, among others, perfumes or pheromones. This very particular equipment allows to create a vortex between 2 reagent solutions injected continuously.

The second work is reported in U.S. Pat. No. 10,071,944B2. This patent reports a way of making aldehydes or acids continuously by means of a tubular continuous reactor by an ozonolysis reaction of unsaturations. Such a method cannot be applied to pheromones carrying multiple bonds that are sensitive to oxidizing conditions.

The academic world reports some works allowing to consider the use of continuous oxidative chemistry to manufacture pheromones. Thus, in V. Liautard & al. Catalysts 2018, 8, 529, the authors report the conversion of ferrugineol, a secondary alcohol into a ketone in the presence of magnesium and a donor aldehyde according to the Oppenauer oxidation reaction. However, apart from the fact that obtaining a ketone is easier than obtaining an aldehyde, the yield in this work is very low since only 10% of alcohol is actually converted into a ketone. Moreover, the structure of the pheromone does not include any chemical functions sensitive to secondary reactions.

A solution conceivable by the person skilled in the art could have consisted of the transposition of the work published in L. Vannoye & al. Volume 357, Issue 4, Mar. 9, 2015, pages 739-746. Indeed, the latter have shown the possibility of transforming primary vinyl or aromatic alcohols using continuous aerobic oxidation in the presence of a catalyst formed from CuOTf or Cu(OTf)₂ in the presence of bipyridine and N-methylimidazole. In the case of linear aliphatic compounds, the catalytic system leads to low conversions incompatible with the purity necessary for pheromones. Moreover, the copper catalyst considered in this publication requires a rate of 5% molar whereas the molecular mass of the catalyst is very high. Without effective recycling of the catalyst, the methods cannot be interesting from an economic point of view but also from their carbon footprint.

This last approach, if it could be applied with a more economical catalyst, would be particularly interesting for the synthesis of pheromones since it would be a question of producing these pheromones by an aerobic oxidation imitating the respiration of living beings.

The applicant has found a novel method which is characterized in that the oxidation reaction is carried out continuously in a reactor under oxygen pressure, advantageously in a H.E.R reactor (Heat Exchange Reactor), such as those marketed by the company Khimod, and that it allows the following reaction to be carried out by recycling the catalyst to limit its impact:

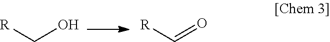

Thus, according to a first embodiment, the invention relates to a method for preparing an aldehyde of general formula (II):

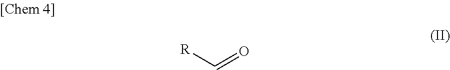

where R is a linear hydrocarbon chain of formula CₙH₂ₙ₋₂ₚ₊₁, with:
n which is a natural number ranging from 9 to 24,
p corresponding to the number of unsaturations of the hydrocarbon chain which is an integer ranging from 1 to 4;

said method is continuous and comprises the following concomitant steps:
a. feeding a continuous reactor under oxygen pressure between 1 and 30 bar with:
   an alcohol of general formula (I):

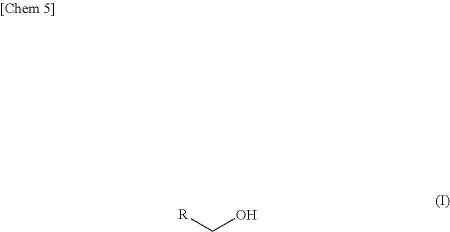

where R, n and p are as defined previously for the compound of formula (II), in solution in an apolar organic liquid phase (A) with a density strictly less than 0.7,
   a copper-based catalyst in solution in a polar liquid phase (B) with a density greater than or equal to 0.75,
the phases (A) and (B) being immiscible with each other,
the alcohol/copper-based catalyst molar ratio ranging from 0.01 to 0.5,
b. recovering the aldehyde in phase (A) by liquid/liquid separation.

According to another embodiment, the method according to the invention is characterized in that the copper-based catalyst further comprises at least one copper ligand of general formula:

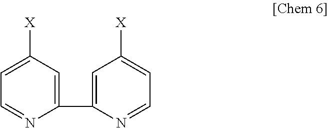

where X is selected from the group consisting of —C(O)—R1, —C(O)O—, —C(O)—OR1, —CF₃, —SO₃R1 and sulfonate —SO₃⁻ and R1 is a linear or branched C1-C8 alkyl group.

Advantageously, the copper-based catalyst further comprises (2,2,6,6-tetramethylpiperidin-1-yl)oxyl (TEMPO) or a derivative such as hydroxy-TEMPO, amino-TEMPO or acetamido-TEMPO.

According to an advantageous embodiment, the method is characterized in that the copper-based catalyst further comprises a base selected from the group consisting of 1,8-diazabicyclo[5.4.0]undec-7-ene (DBU), 1,5-diazabicyclo[4.3.0]non-5-ene (DBN), 1-methylimidazole (NMI) and an acetate salt, in particular sodium acetate or potassium acetate.

Further advantageously, the method according to the invention is characterized in that the copper-based catalyst comprises a bipyridine, in particular 2,2′-bipyridine.

Advantageously, the method is characterized in that the apolar organic liquid phase (A) is selected from the group consisting of a C5-C8 alkane, in particular a linear alkane, more particularly hexane.

According to an advantageous embodiment, the method according to the invention is characterized in that the polar liquid phase (B) is selected from the group consisting of acetonitrile, dimethylsulfoxide (DMSO), sulfolane, a salt of 1-(C1-C6)-alkyl-3-methyl imidazolium and a salt of 1-(C1-C6)-Alkyl-2,3-dimethyl imidazolium, and mixtures thereof.

Advantageously, the counterion of the salt is a fluorinated counterion, in particular selected from trifluoromethylsulfonate (triflate), hexafluorophosphate and tetrafluoroborate.

According to a specific and preferred embodiment, the copper-based catalyst is a copper II salt, advantageously selected from the group consisting of copper II halides and copper II carboxylates.

Advantageously, the copper halide is selected from CuI₂, CuCl₂ and CuBr₂; the copper carboxylate is selected from copper acetate Cu(OAc)₂ and copper II acetyl acetonate Cu(Acac)₂.

According to a particularly advantageous embodiment, the method according to the invention is characterized in that step a) is carried out in a stirred reactor of the heat exchange reactor type.

In a particular embodiment, the continuous oxidation reaction is carried out by co-feeding the continuous reactor with the two solutions, one containing the reactant and the other containing the catalyst.

Thus, in a particular embodiment, the method according to the invention is characterized in that it comprises the following concomitant steps:
a. co-feeding a continuous reactor under oxygen pressure of between 1 and 30 bar with the alcohol of formula (I) in solution in an apolar organic liquid phase (A) of density strictly less than 0.7 and with the copper-based catalyst in solution in a polar liquid phase (B) with a density greater than or equal to 0.75, in order to carry out an oxidation of the alcohol (I);
b. decompressing and liquid/liquid separating the polar liquid phase (B) containing the catalyst and the apolar organic liquid phase (A) containing the product (II);
c. recovering the product (II) in solution in the upper apolar organic liquid phase (A);
d. optionally, evaporating the apolar organic liquid phase (A) to recover the product (II).

According to the embodiment comprising the co-feeding of the continuous reactor with the two solutions, the method is also characterized in that all or part of the polar liquid phase (B) containing the catalyst separated at step b) is reintroduced at the co-feeding step a).

Also advantageously, the embodiment comprising the co-feeding of the reactor with the two solutions is characterized in that the molar ratio between the compound (I) and the copper-based catalyst is between 10:1 and 20:1 at the co-feeding step a).

In an alternative embodiment of the method according to the invention, provision is made for the preparation of a homogeneous two-phase mixture comprising the alcohol of formula (I) in solution in an apolar organic liquid phase (A) with a density strictly less than 0.7 and the copper-based catalyst in solution in a polar liquid phase (B) with a density greater than or equal to 0.75.

The two phases are not miscible, so it is this two-phase mixture which will feed a continuous reactor under oxygen pressure within which the continuous oxidation of the alcohol (I) is carried out.

According to this particular embodiment providing for the preparation of a homogeneous two-phase preliminary mixture, a recirculation loop is provided between the mixer M in which the homogeneous two-phase mixture is kept stirred and the continuous reactor under pressure in which the reaction of oxidation takes place. This recirculation loop allows to deplete the medium of alcohol (I) and enrich it with the formed aldehyde (II); this recirculation loop operates until the two-phase mixture is substantially depleted of the alcohol (I), that is to say it is substantially completely oxidized to the aldehyde (II).

The expression “homogeneous two-phase mixture”, here means that stirring in the mixer allows to create and maintain a mixture in which the two phases (A and B), although immiscible, are uniformly distributed in each other and are not distinguishable with the naked eye and are not separated within the mixer. A homogeneous two-phase mixture is characterized by the fact that if a sample is taken from said mixture, it contains a substantially equal amount of both phases.

Thus in the case of an embodiment of the method according to the invention with the preparation of a homogeneous two-phase mixture, said method is characterized in that it comprises the following concomitant steps:
a. preparing, in a mixer M, a homogeneous two-phase mixture comprising the alcohol of formula (I) in solution in an apolar organic liquid phase (A) of density strictly less than 0.7 and the copper-based catalyst in solution in a polar liquid phase (B) with a density greater than or equal to 0.75;
b. feeding a continuous reactor under oxygen pressure of between 1 and 30 bar with the homogeneous two-phase mixture to carry out the oxidation of the alcohol (I);
c. creating a recirculation loop between the stirred reactor and the mixer M until substantially complete conversion of the alcohol (I) into aldehyde (II);
d. decompressing and liquid/liquid separating the polar liquid phase (B) containing the catalyst and the apolar organic liquid phase (A) containing the product (II);
e. recovering the product (II) in solution in apolar organic liquid phase (A);
f. optionally, evaporating the apolar organic liquid phase (A) to recover the product (II).

According to a particular embodiment of the method according to the above embodiment, with preparation of a homogeneous two-phase mixture, the oxygen which has not reacted in the continuous reactor, in particular a continuous H.E.R reactor (Heat Exchange Reactor) is decompressed at the outlet of said reactor, captured, recompressed and reinjected into said reactor at the feed.

According to a particular embodiment of the method according to one of the embodiments as described above, the continuous reactor is a HER reactor (Heat Exchange Reactor).

The method according to the invention is characterized in that it is carried out continuously in a polar solvent in the presence of an inexpensive copper-based catalyst under an air pressure of more than 1 bar and less than 30 bar, and at a temperature of between 30 and 200° C., in particular of between 40 and 180° C. The residence time in the oxidation reactor is advantageously less than 240 minutes, more advantageously the residence time is between 5 min and 80 minutes.

This method offers the advantage of a high selectivity of the reaction at reduced costs.

The complete continuous solution oxidation method generally comprises three main steps or zones:
Step/Zone 1: catalyst and alcohol preparation step;
Step/Zone 2: alcohol oxidation step (I);
Step/Zone 3: aldehyde recovery step (II).

### Step/Zone 1: Preparation of the Catalyst

The catalyst used is obtained by mixing an equivalent of a copper (II) salt such as copper halides, in particular CuI₂, copper triflate, copper acetate, copper acetyl acetonate, copper hydroxide with a dense polar solvent or mixture of solvents, in order to obtain a polar liquid phase (B) with a density greater than or equal to 0.75, in particular greater than or equal to 0.8, or even greater than or equal to 0.9 or even greater than or equal to 1.

Suitable solvents are acetonitrile, dimethylsulfoxide (DMSO), sulfolane, 1-(C1-C6)-alkyl-3-methylimidazolium salt and 1-(C1-C6)-alkyl-2,3-dimethyl imidazolium, and mixtures thereof. And advantageously, the counterion of the salt is a fluorinated counterion, in particular selected from trifluoromethylsulfonate (triflate), hexafluorophosphate and tetrafluoroborate acetonitrile or preferably ionic liquids such as salts of 1-alkyl,3 methyl imidazolium or 1-alkyl-2,3-dimethyl imidazolium.

The catalyst thus obtained is at a concentration of between 0.01M and 1M.

Can be added, between 1 and 4, even between 1.8 and 2.5 molar equivalent of ligand of general formula:

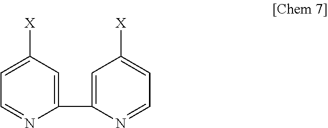

where X is selected from the group consisting of —C(O)—R1, —C(O)O—, —C(O)—OR1, —CF3, —SO3R1 and sulfonate —SO₃⁻ and R1 is a linear or branched C1-C8 alkyl group.

Of more than 0.5 to 2 equivalent, in particular 1 equivalent, of (2,2,6,6-tetramethylpiperidin-1-yl)oxyl (TEMPO), or a derivative thereof may be added.

Finally, between 0 and 4 equivalents, in particular between 1 and 3, or even between 1 and 2.2 equivalents of a base selected from the group consisting of 1,8-diazabicyclo[5.4.0]undec-7-ene (DBU), 1,5-diazabicyclo[4.3.0]non-5-ene (DBN), 1-methylimidazole (NMI) and an acetate salt, in particular a sodium acetate or a potassium acetate.

The mixture containing the alcohol (I) is produced in a conventional manner by mixing the alcohol (i) in an apolar organic solvent so as to obtain an apolar organic liquid phase (A) with a density strictly less than 0.7, or even less than 0.6. The concentration of the alcohol in said apolar organic liquid phase is between 0.1M and 10M, preferably between 0.1 and 1M.

### Step/Zone 2: Alcohol Oxidation Step (I)

As mentioned previously, the continuous oxidation step can be carried out in two different manners/ways. Either by co-feeding a continuous reactor with the two phases, or by feeding said continuous reactor with a homogeneous two-phase mixture previously prepared and kept stirring in a mixer. The method can be implemented in 2 ways:

### Way No 1:

The catalyst mixture is prepared in a stirred reactor or in a reaction tube as mentioned above, then pumped towards the continuous reaction zone in a continuous reactor, in particular of the H.E.R. (for Heat Exchange Reactor) type. At the inlet of the continuous reactor, the phase containing the alcohol (I) and the oxygen are also injected under a pressure of between 1 and 30 bars. Such an embodiment is illustrated in FIG. 1.

The injections are controlled by flowmeters at flows denoted F_{newcata}, F_{substrate} and F_{O2} so that the conversion at the output of the H.E.R. is greater than 99%.

The reaction takes place in the continuous reactor at a temperature of between 20° C. and 200° C. and the reaction product arrives in zone 3 for separating the catalyst, the gases and the solution containing the aldehyde (II).

The continuous reactor is continuously fed with the phase containing the alcohol (I), the phase containing the catalytic system and oxygen.

The reinjected catalyst flow (F_{recyccata}) is done in the fresh catalyst flow and the two flows are controlled so that the molarity between the alcohol substrate (I) and the catalyst (combining the fresh catalyst flow and the flow of recycled catalyst) is maintained at a ratio of between 10/1 and 20/1. When the flows F_{substrate} and F_{O2} are constant then F_{newcata}+F_{recyccata} is constant. Consequently, the rate of catalyst is controlled by 3 UV detectors: UV1 allows to know the concentration of fresh catalyst, UV3 allows to know the concentration of recycled catalyst, UV2 allows to check that the desired concentration of catalyst is indeed injected continuously into the reactor.

It will be recalled that in a continuous reactor, the concentration in the reaction medium corresponds to the concentration at the reactor outlet.

The operating pressure of the reactor is advantageously between 1 bar and 200 bars, more advantageously between 1 and 100 bars. In certain embodiments, the operating pressure of the reactor is between 1 bar and 50 bars.

The oxidation temperature is advantageously between 15 and 100° C., particularly between 20 and 80° C. The oxidation temperature is of course lower than the decomposition temperature of the product.

The oxidation temperature is advantageously kept constant. Any means known to the person skilled in the art can be used for this purpose. By way of example, mention can be made of the heat exchanger inside the reactor, outside, by controlling the feed temperature.

The continuous reactor can advantageously be equipped with stirring means such as static mixers. Indeed, sufficient stirring allows to guarantee a good level of mixing and thus to avoid dead zones or segregation of the reaction medium.

Preferably, the continuous reactor is a continuous reactor of the HER (Heat Exchange Reactor) type as marketed by the company Khimod.

By definition, a continuous reactor has at least one inlet and at least one outlet which are systematically open. As known to the person skilled in the art, the outlet of the reactor must be far enough from the inlet to avoid preferential path problems. Ideally, the inlet and outlet of the reactor are as far apart as possible.

In the case of a two-phase reactor, the outlet is of course placed in contact with the liquid phase.

The effluent from the stirred reactor is sent to the oxidation product recovery step.

### Step/Zone 3: Aldehyde Recovery Step (II)

This zone 3 is characterized first by a decompressor which evacuates the oxygen allowing its optional recycling, then by a continuous decanter which pumps the denser solution containing the catalyst from the bottom of the decanter and sends by overflow to zone/step 1 passing through a (thermal or chemical) desiccator.

The recovery of the aldehyde from the solution and the separation of its solvent can be done according to any method known to the person skilled in the art, so as to isolate it and bring it to a level of volatile matter lower than 1% by weight.

### Way No 2:

A second way of implementing the method comprises the production of a homogeneous two-phase mixture comprising on the one hand the catalyst in one phase, and on the other hand the alcohol reactant (I) in another phase, the two phases not being miscible. Such an embodiment is illustrated in FIG. 2.

In this case, a mixture of solutions of the catalyst and of the reagent R—CH₂OH is prepared in a mixer, itself connected to the continuous reactor, for example a continuous reactor of the H.E.R. type, by a circulation loop at a flow rate D1. Upstream of the continuous reactor, for example a continuous reactor of the HER type, the oxygen is introduced at a flow rate D2 and at a pressure of between 0.2 and 30 bars. The unreacted oxygen is decompressed at the outlet of the continuous reactor to be optionally recycled at the inlet of the continuous reactor, for example reactor of the H.E.R. type. The continuous reactor is kept under stirring and the recirculation loop operates until total conversion of alcohol (I).

In this embodiment, the temperatures of the mixer and of the continuous reactor, in particular a H.E.R. reactor, are preferably the same, comprised and preferably between 15 and 80° C., in particular between 20 and 60° C.

The advantage of the method according to way 1 or way 2 is, on the one hand, to allow complete conversion of an alcohol into an aldehyde, in particular a pheromone bearing the aldehyde function with a very satisfactory degree of purity. On the other hand, these two ways of proceeding allow to avoid having recourse to reactors of large volume withstanding high pressure, the phase under gas pressure being limited to the part of the reaction taking place in the continuous reactor under pressure, in particular a HER-type reactor.

### LEGEND OF FIGURES

FIG. 1: implementation of the method with co-feeding the reactor with the two phases.

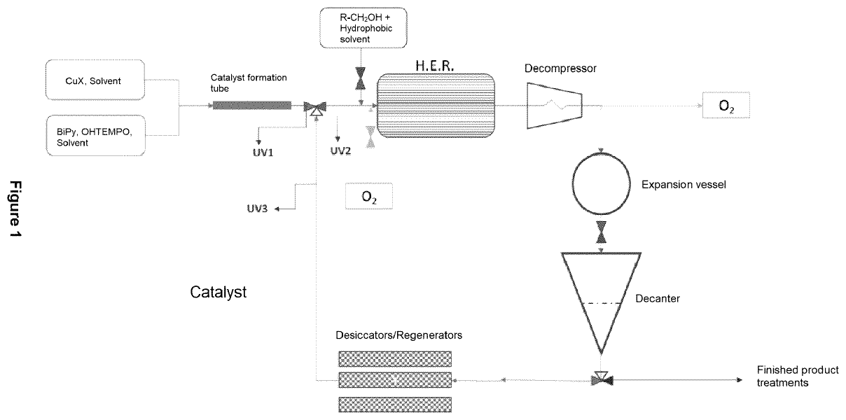

FIG. 2: implementation of the method with prior preparation of a homogeneous two-phase mixture in a mixer continuously feeding a reactor, with a recirculation loop between the reactor and the mixer.

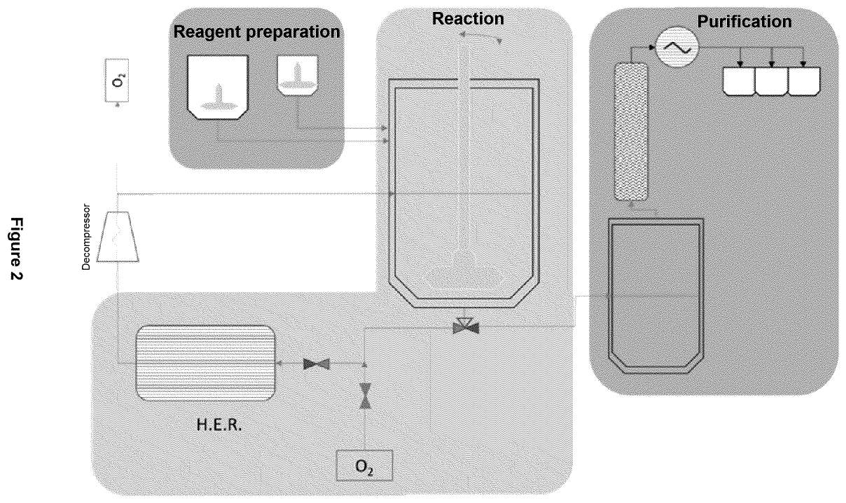

### EXAMPLES

The raw materials (CuI₂, bipyridine, TEMPO) and solvents are found commercially at Sigma Aldrich.

Z11-hexadecenol is produced according to a method known to the person skilled in the art on the Salin de Giraud (M2I Development) site and has a purity of 92% by weight. The main impurity (3.2%) is E11-hexadecenal.

The H.E.R. reactor is manufactured and supplied by the Khimod company.

### Example 1: Embodiment with Preparation of a Homogeneous Two-Phase Mixture

In a 10 L reactor kept under vigorous stirring, 480 g of Z11-hexadecenol are prepared in 3.3 L of hexane then 3 L of an acetonitrile solution containing:
19 g of copper iodide (CuI₂),
15.6 g of bipyridine,
16.4 g of N-methyl imidazole,
15.6 g of TEMPO.

The two-phase mixture is stirred so as to have a homogeneous distribution of the two phases.

The solution is pumped towards the HER by means of a high pressure pump at a rate of 50 mL/min at the same time as oxygen which is introduced at 12 bars at a rate of 1 L/min. The entire system is maintained at 25° C.

Regular samples are taken from the 1 L reactor and the reaction is stopped when the conversion of the Z11-hexadecenol is complete after 10 hours.

At the end of the reaction, the recirculation is stopped as well as the stirring. The lower phase is evacuated to be optionally recycled (cf. example 3). The upper phase is kept in the reactor, washed twice with distilled water then the solvent is evaporated under vacuum to recover 456 g of Z11-hexadecenal (purity 92.0%). It is interesting to note that in the initial product 3.4% of E11-hexadecenol was present and that 3.5% of E11-hexadecenal is found in the final product.

### Example 2: Embodiment with Co-Feeding the Reactor with the Two Phases, without Recycling the Catalyst

In a 5 L reactor kept under vigorous stirring, 0.564 L of Z11-hexadecenol in 1.436 L of hexane (concentration of 1 Mol/L) is prepared.

In another 5 L reactor, 2 L of an acetonitrile solution containing:
19 g of copper iodide (CuI₂),
15.6 g of bipyridine,
16.4 g of N-methyl imidazole,
15.6 g of TEMPO.

The two solutions are pumped by means of HPLC pumps into the H.E.R. reactor at flow rates of 4.2 mL/min for each solution. The molar ratio between copper catalyst and alcohol is now 0.02. The oxygen is introduced at 12 bars at a flow rate of 0.2 L/min. And the reaction product is recovered after decompression in a 10 L separating funnel type decanter. The residence time is 2 hours for a total reaction time of 4 hours. At the end the two phases are separated (bluish phase containing the catalyst at the bottom), then the organic phase is washed until there is total discoloration. The hexane is evaporated and 460 g of hexadecenal with a purity of 93% by weight are obtained.

The results are similar to those of Example 1.

### Example 3: Embodiment with Co-Feeding the Reactor with the Two Phases and with Recycling the Catalyst

In a 5 L reactor kept under vigorous stirring, 0.564 L of Z11-hexadecenol in 1.436 L of hexane (concentration of 1 Mol/L) is prepared.

In another 2 L reactor, 1 L of a solution of 1-butyl-2,3-dimethyl imidazolium hexafluorophosphate containing:
38 g of copper iodide (CuI₂),
31.2 g of bipyridine,
32.8 g of N-methyl imidazole,
31.2 g of TEMPO.

The two solutions are pumped by means of HPLC pumps into the H.E.R. reactor. The flow rate of the reagent solution is 42 mL/min for the 2 reagents. The molar ratio between copper catalyst and alcohol is now 0.22.

The reaction product is recovered after decompression in a 10 L separating funnel type decanter. The lower phase is itself pumped continuously to resupply the catalyst reserve.

Oxygen is introduced at 12 bars at a flow rate of 2 L/min.

The residence time is 24 minutes for a total reaction time of 48 min. After washing and evaporation of the hexane, 447 g of Z11-hexadecenal with a purity of 91.8% by weight are recovered.

### Example 4: Embodiment with Co-Feeding the Reactor with the Two Phases and with Recycling the Catalyst

In a 50 L reactor kept under vigorous stirring, 5.6 L of Z11-hexadecenol are prepared in 14 L of hexane (concentration of 1 Mol/L).

In another 2 L reactor, 1 L of a solution of 1-butyl-2,3-dimethyl imidazolium hexafluorophosphate is prepared containing:
38 g of copper iodide (CuI₂),
31.2 g of bipyridine,
32.8 g of N-methyl imidazole,
31.2 g of TEMPO.

The two solutions are pumped by means of HPLC pumps into the H.E.R. reactor. The flow rate of the reagent solution is 42 mL/min for the 2 reagents. The molar ratio between copper catalyst and alcohol is 0.22. And the reaction product is recovered after decompression in a 10 L separating funnel type decanter. The lower phase is itself pumped continuously to resupply the catalyst reserve. The organic phase is regularly pumped from the top of the funnel into a 50 L buffer tank.

The oxygen is introduced at 12 bars at a flow rate of 2 L/min.

The residence time is 24 minutes for a total duration of total reaction of 8 hours.

After washing and evaporation of the organic phases, 4.56 kg of Z11-hexadecenal at 92.3% by weight are obtained.

1. A method for preparing an aldehyde of general formula (II): 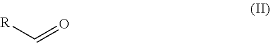
wherein R is a linear hydrocarbon chain of formula CₙH₂ₙ₋₂ₚ₊₁,
wherein:
n is a natural number ranging from 9 to 24;
p corresponds to the number of unsaturations of the hydrocarbon chain which is an integer ranging from 1 to 4;
wherein the method is continuous and comprises the following concomitant steps:
a) feeding a continuous reactor under an oxygen pressure of between 1 and 30 bar with:
i) an alcohol of general formula (I): 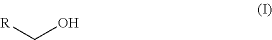
wherein R is as defined previously for the compound of formula (II), in solution in an apolar organic liquid phase (A) with a density strictly less than 0.7;
ii) a copper-based catalyst in solution in a polar liquid phase (B) with a density greater than or equal to 0.75;
the phases (A) and (B) being immiscible with each other,
the alcohol/copper-based catalyst molar ratio ranging from 0.01 to 0.5;
b) recovering the aldehyde in phase (A) by liquid/liquid separation.

2. The method according to claim 1, wherein the copper-based catalyst further comprises at least one copper ligand of general formula: 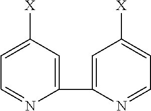
wherein X is selected from the group consisting of —C(O)—R1, —C(O)O—, —C(O)—OR1, —CF₃, —SO₃R1 and sulfonate —S₃⁻; and
R1 is a linear or branched C1-C8 alkyl group.

3. The method according to claim 1, wherein the copper-based catalyst further comprises (2,2,6,6-tetramethylpiperidin-1-yl)oxyl (TEMPO) or a derivative thereof.

4. The method according to claim 1, wherein the copper-based catalyst further comprises a base selected from the group consisting of 1,8-diazabicyclo[5.4.0]undec-7-ene-(DBU), 1,5-diazabicyclo[4.3.0]non-5-ene (DBN), 1-methylimidazole (NMI) and an acetate salt.

5. The method according to claim 1, wherein the copper-based catalyst comprises a bipyridine.

6. The method according to claim 1, wherein the apolar organic liquid phase (A) is selected from the group consisting of C5-C8 alkanes.

7. The method according to claim 1, wherein the polar liquid phase (B) is selected from the group consisting of acetonitrile, dimethylsulfoxide (DMSO), sulfolane, a salt of 1-(C1-C6)-alkyl-3-methyl imidazolium, a salt of 1-(C1-C6)-alkyl-2,3-dimethyl imidazolium, and mixtures thereof.

8. The method according to claim 7, wherein the salt of 1-(C1-C6)-alkyl-3-methyl imidazolium is 1-(C1-C6)-alkyl-3-methyl imidazolium trifluoromethylsulfonate, 1-(C1-C6)-alkyl-3-methyl imidazolium hexafluorophosphate, or 1-(C1-C6)-alkyl-3-methyl imidazolium tetrafluoroborate; and the salt of 1-(C1-C6)-alkyl-2,3-dimethyl imidazolium is 1-(C1-C6)-alkyl-2,3-dimethyl imidazolium trifluoromethylsulfonate, 1-(C1-C6)-alkyl-2,3-dimethyl imidazolium hexafluorophosphate or 1-(C1-C6)-alkyl-2,3-dimethyl imidazolium tetrafluoroborate.

9. The method according to claim 1, wherein the copper-based catalyst is a copper II salt.

10. The method according to claim 9, wherein the copper-based catalyst is selected from the group consisting of CuI₂, CuCl₂, CuBr₂, Cu(OAc)₂ and Cu(Acac)₂.

11. The method according to claim 1, wherein the step a) is carried out in a continuous reactor of the heat exchange reactor type.

12. The method according to claim 1, further comprising the following concomitant steps:
a. co-feeding a continuous reactor under an oxygen pressure of between 1 and 30 bar with the alcohol of formula (I) in solution in the apolar organic liquid phase (A) of density strictly less than 0.7 and with the copper-based catalyst in solution in the polar liquid phase (B) with a density greater than or equal to 0.75, in order to carry out an oxidation of the alcohol of formula (I) into the aldehyde of general formula (II);
b. decompressing and liquid/liquid separating the polar liquid phase (B) containing the catalyst and the apolar organic liquid phase (A) containing the aldehyde of general formula (II);
c. recovering the aldehyde of general formula (II) in solution in the apolar organic liquid phase (A) which is in an upper phase;
d. optionally, evaporating the apolar organic liquid phase (A) to recover the aldehyde of general formula (II).

13. The method according to claim 12, wherein all or part of the polar liquid phase (B) containing the copper-based catalyst is reintroduced at the co-feeding step a).

14. The method according to claim 12, wherein the molar ratio between the alcohol of general formula (I) and the copper-based catalyst is between 10:1 and 20:1 at the co-feeding step.

15. The method according to claim 1, further comprising following concomitant steps:
a. preparing, in a mixer M, a homogeneous two-phase mixture comprising the alcohol of formula (I) in solution in the apolar organic liquid phase (A) of density strictly less than 0.7 and the copper-based catalyst in solution in the polar liquid phase (B) with a density greater than or equal to 0.75;
b. feeding a continuous reactor under an oxygen pressure of between 1 and 30 bar with the homogeneous two-phase mixture in order to carry out the oxidation of the alcohol of formula (I) into the aldehyde of general formula (II);
c. creating a recirculation loop between the continuous reactor under stirring and the mixer M until substantially complete conversion of the alcohol of formula (I) into the aldehyde of general formula (II);
d. decompressing and liquid/liquid separating the polar liquid phase (B) containing the catalyst and the apolar organic liquid phase (A) containing the aldehyde of general formula (II);
e. recovering the aldehyde of general formula (II) in solution in the apolar organic liquid phase (A);
f. optionally, evaporating the apolar organic liquid phase (A) to recover the aldehyde of general formula (II).

16. The method according to claim 15, wherein oxygen which has not reacted in the continuous reactor is decompressed at outlet of the continuous reactor, captured, recompressed and reinjected into the continuous reactor at feed.

17. The method according to claim 3, wherein the derivative of (2,2,6,6-tetramethylpiperidin-1-yl)oxyl (TEMPO) is hydroxy-TEMPO, amino-TEMPO or acetamido-TEMPO.

18. The method according to claim 4, wherein the acetate salt is sodium acetate or potassium acetate.

19. The method according to claim 6, wherein the apolar organic liquid phase (A) is hexane.

20. The method according to claim 9, wherein the copper-based catalyst is selected from the group consisting of copper II halides and copper II carboxylates.
